(19) United States
(12) Patent Application Publication
(10) Pub. No.: US 2008/0314391 A1
(43) Pub. Date: Dec. 25, 2008

(54) Portable "cleaned, decontaminated and disinfected air" breathing device

(76) Inventor: Nand K. Acharya, Los Angeles, CA (US)

Correspondence Address: NAND K. ACHARYA, APT. 104-335, 7095 HOLLYWOOD BLVD., LOS ANGELES, CA, 90028, US

(21) Appl. No.: 11/789,056

(22) Filed: Apr. 23, 2007

(51) Int. Cl. A61M 15/08; A62B 18/02

(52) U.S. Cl. 128/207.18; 128/205.25

(57) ABSTRACT

Present invention relates to protect the wearer from airborne viruses like influenza, and all harmful bacteria. This device will allow a user to carry out daily chorus such as eating, drinking, walking, talking and driving without taking it off. This device allows the user doing all the daily activity while filtering out all the viruses and harmful bacteria.

### STATEMENT AS TO RIGHTS TO INVENTIONS MADE UNDER FEDERALLY SPONSORED RESEARCH AND DEVELOPMENT

Not Applicable

### BACKGROUND OF INVENTION

### Field of Invention

This invention nasal cannula and liquid container, more particularly, a nasal cannula and liquid container for delivering a clean and decontaminated air to the person.

Clean and virus free air is absolutely necessary to human health. Each person require a steady dose of clean air. When a person's air is not clean or saturated with harmful bacteria or viruses, that person becomes ill and may be even die without proper medication.

Known masks and respirators and air filters covers a person's nose and mouth. These respirators are bulky and have to be taken off while eating or drinking and risks catching infections. A need, therefore, exists for the kind of device which does not to be taken off and continues to allow clean air while eating, drinking, talking, therefore, a need exists for a clean and decontaminated air delivery device.

### SUMMARY OF THE INVENTION

In accordance with one aspect of the invention, in a preferred embodiment, the nasal cannula is a hollow tubular member having two ends and one end having two identical features.

(A) A hollow tube in the middle for breathing in the clean air through the container's medicated liquid.
(B) A porous tube around for to exhale without removing it from nostrils.
(C) And the top skin to guide away the exhaled air.

Opposite end of the tube is connected to liquid container. Further the clean air supply tube is position above and behind the ears of the user.

In accordance with yet another aspect of invention, in carrying out the invention, the assembly is preferably made of a flexible elastomeric material. However, the assembly can be of any suitable lightweight, flexible and comfortable material as known in the art.

In accordance with yet still another aspect of the invention a method of delivering clean and decontaminated air to a person by applying nasal cannula and a container is also provided.

In accordance with yet another aspect of the invention, the container, in carrying out the invention, the assembly is preferably made of light weight plastic, the assembly can be of any suitable lightweight and compatible material as known in the art.

Numerous advantages may be realized by the present invention. For example, the assembly overcomes the drawbacks of known respirators and delivers a steady dose of clean and

decontaminated air to a person's nasal without any discomfort.

Other advantages and features of the invention will become apparent from the following description and from the reference to the drawing.

User will pour the water into the container by unscrewing the top 1 FIG. 1. The user will wear the device around the neck as shown in FIG. 1 or across the shoulder. Specially designed cannula 14 and 15 as shown in FIG. 4 will sit inside nostrils as shown in FIG. 1. Each time the user breaths in the air will be forced in through ventilations X and Y at the bottom of the chamber as shown 2 and 3 in FIG. 1. From the bottom of the water/liquid air will rise leaving all the impurity and contaminants in water/liquid, will make its way to the tube 8, as shown by the arrows, through the tube to nasal cannula.

This nasal cannula is specially designed to sit inside the nostrils. The conventional nasal cannula tube has been lengthened and a porous layer has been built to facilitate exhaling/breathing out and a sleeve has been put on the top, so it will guide exhaled air without need of cannula being taken out. Each time the user breaths in the air will only come in through 14 and 19, FIG. 4, and will breathe out through porous part 15 and 18 and out under the sleeves 16 and 17.

When the user inhales/breaths in, the sleeves 16 and 17 is tight enough to prevent the air coming and therefore it will only come through ventilations X and Y to the bottom of the water/liquid 2 and 3 to 8 and the air will come out only through the porous part 15 and 18 under the sleeves. With 6 and 7 tube and container are connected. Strap 13 is not the part of my invention.

“Strap 13 is not the part of my invention.” has been deleted

### BRIEF DESCRIPTION OF DRAWINGS

FIG. 1 Shows the way the user will wear the device around his/her neck below the chin.

FIG. 2 is a front view of the device.

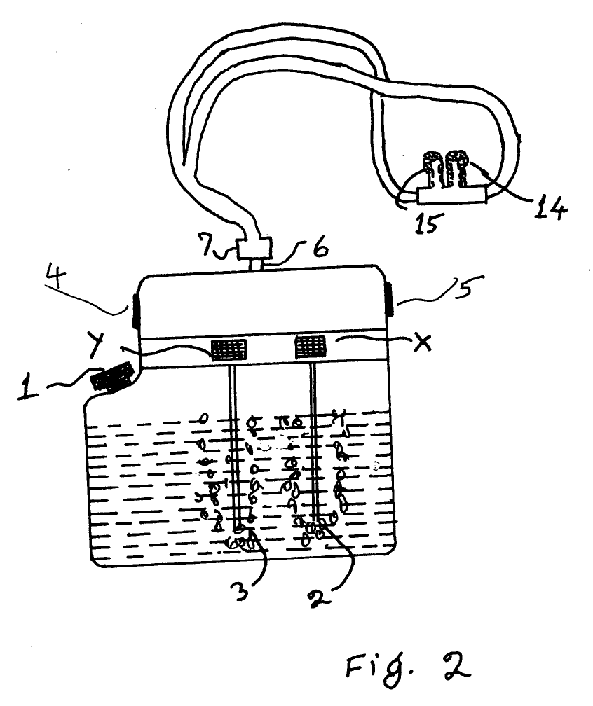

FIG. 3 is a perspective view of the device without the tube.

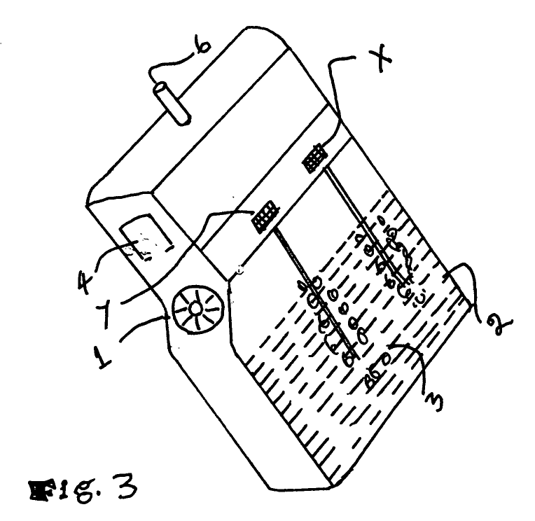

FIG. 4 is a perspective view of the tube which connects the container to nostrils.

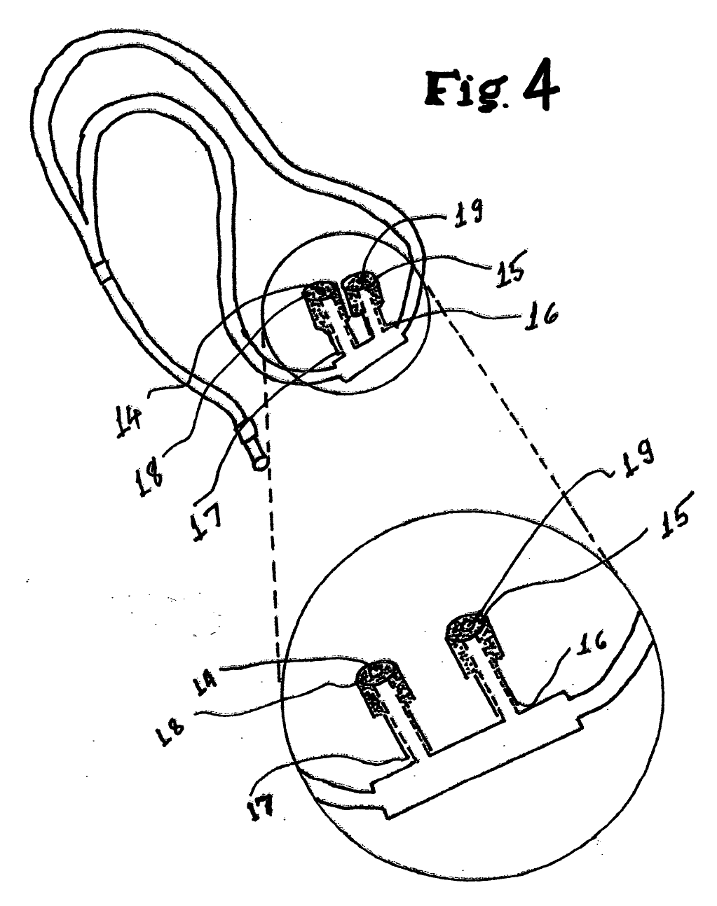

FIG. 5 is the top plan view of the container.

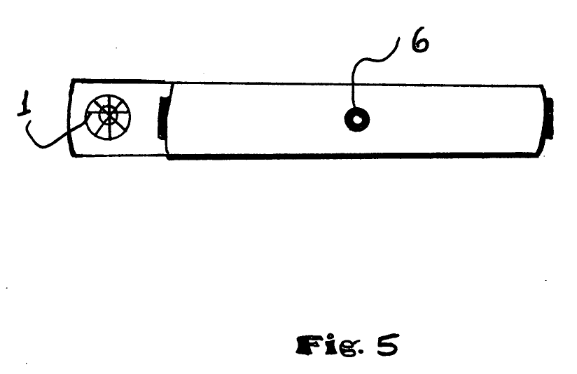

FIG. 6 is an elevation view of the side of the container.

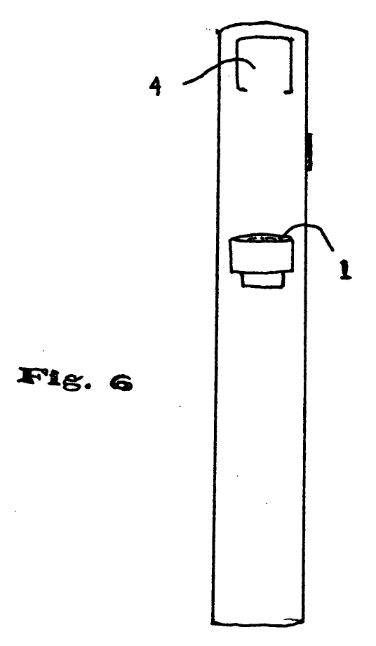

FIG. 7 is another elevation view of the side of the container.

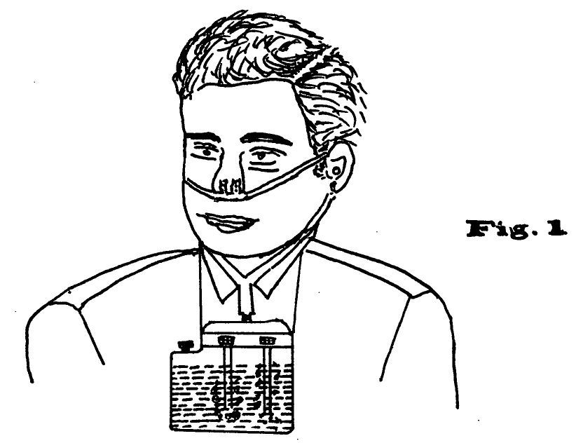

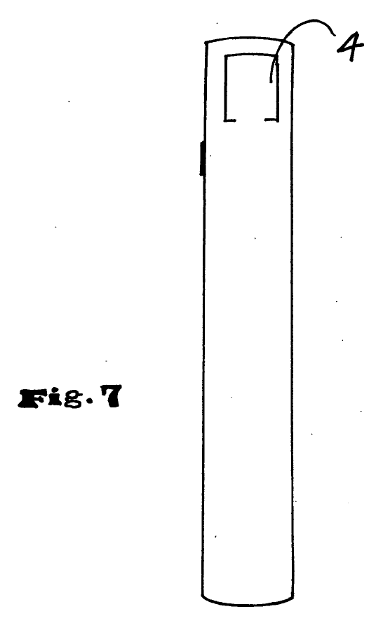

1) This device will be displayed on, in front of the user below the chin As shown on the FIG. 1 or across the shoulder.

2) Once the new designed two-way nasal connula is put on as shown on the front page, the user can breathe in and breathe without taking it off.

3) The user will breathe in only through the clean water or Hydrogen Peroxide Solution.

4) Each time the user breaths in, the air will be forced into the water through the X and Y ventilations leaving all the impurities in the water before the air reaches the lungs of the user.

7) Air entering through ventilations X and Y will leave all the airborne Pollen in the water which will benefit the hey fever sufferers.

8) During the Influenza Pandemic air borne viruses will be killed while Passing through Hydrogen Peroxide Solution mixed water, before Inhaling by the user, save his or her life.

9) Air borne T. B. virus will washed out and killed while traveling/passing through Hydrogen Peroxide Solution.

11) User will be able to talk, walk, drive, eat and drink while this Device is on as shown in FIG. 1.

12) User can put some water based fragrant in the chamber and enjoy.
